(19) United States
(12) Patent Application Publication
(10) Pub. No.: US 2024/0091411 A1
(43) Pub. Date: Mar. 21, 2024

(54) EAR CARTILAGE TISSUE ENGINEERING COMPLEX AND USE THEREOF

(71) Applicant: SHANGHAI RESTHETIC BIO CO., LTD, Shanghai (CN)

(72) Inventors: Yu LIU, Shanghai (CN); Guangdong ZHOU, Shanghai (CN); Zheng CI, Shanghai (CN)

(21) Appl. No.: 18/262,224

(22) PCT Filed: Jan. 17, 2022
(86) PCT No.: PCT/CN2022/072409

§ 371 Date: Jul. 20, 2023

(30) Foreign Application Priority Data
Jan. 20, 2021 (CN) 202110075631.X

(51) Int. Cl. A61L 27/38; A61L 27/36; A61L 27/56

(52) U.S. Cl. CPC A61L 27/3817; A61L 27/3608; A61L 27/3633; A61L 27/3691; A61L 27/3852; A61L 27/3895; A61L 27/56; A61L 2430/24

(57) ABSTRACT

An ear cartilage tissue engineering complex and a preparation method therefor are provided. The complex includes an ear cartilage gel or ear cartilage patch particles containing ear cartilage cells, and a porous frame structure. The ear cartilage gel or ear cartilage patch particles are inoculated onto the porous frame structure to form an ear cartilage gel/ear cartilage patch particle-frame structure complex. The ear cartilage tissue engineering complex is used for the repair and reconstruction of articular cartilage.

### TECHNICAL FIELD

The present invention relates to the field of biomedical tissue engineering, in particular to an ear cartilage tissue engineering complex and preparation method thereof, and its use in repairing joint defects.

### BACKGROUND

In recent years, with the rapid development of economy and society, various types of articular cartilage lesions such as various sports injuries, accidental injuries, and joint degenerative diseases have become more and more common, and cartilage lesions are difficult to repair depending on the patient's self-ability, so there is a huge clinical demand for joint repair. At present, the clinical treatment methods for articular cartilage lesions are mainly palliative treatment and restorative treatment. Palliative treatment mainly includes arthroscopic debridement and chondroplasty. This type of treatment can clean the uneven cartilage surface of the joint surface and remove cartilage fragments, etc., to restore the smooth and flat joint surface. This method is less traumatic and can relieve the patient's symptoms to a certain extent, but its curative effect is limited and cannot effectively relieve the development of arthritis. Restorative treatment includes microfracture treatment, osteochondral transplantation, etc. Although this kind of treatment can repair focal articular cartilage defects to a certain extent, the larger trauma can easily lead to complications in the donor area. In general, traditional treatment methods have poor curative effect. Autotransplantation lacks donors, and long-term complications of artificial joints are difficult to avoid.

With the progress of tissue engineering, people have gradually begun to study the use of scaffolds or tissues constructed by tissue engineering to try to repair cartilage defects. The in situ chondrocyte-material complex constructed by the existing tissue engineering repair technology (autologous chondrocyte implantation (ACI) or matrix-induced autologous chondrocyte implantation (MACI)) has been gradually developed in clinical treatment, but there are still many disadvantages that are difficult to avoid: 1. The source of the cells is articular cartilage. Obtaining cells from the damaged joints will inevitably increase the damage of the original joints. 2. The proliferation ability of articular chondrocytes is limited. The construction cycle of early cell-material complex is long, and patients need to wait for a long time. 3. The strength of the cell-material complex is low, which is far less than the mechanical strength level of natural cartilage.

Therefore, new methods of joint repair are urgently needed.

### SUMMARY OF THE INVENTION

The purpose of the present invention is to provide an ear cartilage tissue engineering complex, a preparation method thereof, and its use in repairing joint defects.

The first aspect of the present invention provides an ear cartilage tissue engineering complex, which comprises:
(a) a carrier comprising a porous frame structure; and
(b) ear cartilage gel or ear cartilage sheet pieces that are inoculated on or loaded on the carrier.

In another preferred embodiment, the complex comprises a complex formed by inoculating the ear cartilage gel or ear cartilage sheet pieces on the carrier and undergoing chondrogenic culture (in which the ear chondrocytes are loaded on the carrier and form a more closely integrated structure with the carrier).

In another preferred embodiment, the complex comprises a complex formed by inoculating the ear cartilage gel or ear cartilage sheet pieces on the carrier without chondrogenic culture.

In another preferred embodiment, the ear chondrocytes are derived from a human or non-human mammal.

In another preferred embodiment, the ear chondrocytes are derived from autologous ear chondrocytes or xenogenous ear chondrocytes, preferably autologous ear chondrocytes.

In another preferred embodiment, the ear chondrocytes are taken from the subject's autologous ear chondrocytes.

In another preferred embodiment, the subject is a human or a non-human mammal.

In another preferred embodiment, the subject has a joint defect.

In another preferred embodiment, the joint defect is an articular cartilage defect.

In another preferred embodiment, the joint defect is a knee joint defect, an elbow joint defect, a hip joint defect, an ankle joint defect, a wrist joint defect, a mandibular joint defect, and a combination thereof.

In another preferred embodiment, the ear cartilage gel comprises a cell population of chondrocytes and extracellular matrix secreted by chondrocytes, wherein the extracellular matrix encapsulates the cell population, and the ear cartilage gel is in a gel state, and the density of chondrocytes is at least 1.0×10⁸ cells/ml or 1.0×10⁸ cells/g.

In another preferred embodiment, the ear cartilage gel is prepared by gelation culture of ear chondrocytes. In another preferred embodiment, the gelation culture is an in vitro culture with gelation medium.

In another preferred embodiment, the gelation medium contains the following components: high-glucose DMEM medium containing 4 to 5 wt % glucose, 10% FBS (v/v) and 100 U/ml penicillin-streptomycin.

In another preferred embodiment, the adhesion rate of the ear cartilage gel is ≥90%, preferably ≥95%.

In another preferred embodiment, in the ear cartilage gel, the concentration of chondrocytes is 1.0×10⁸ cells/ml-10×10⁸ cells/ml, preferably 1.5-5×10⁸ cells/ml.

In another preferred embodiment, the ear cartilage gel is obtained by gelation culture for 2.5-5.5 days, preferably for 3-5 days.

In another preferred embodiment, the ear chondrocytes are derived from a human or non-human mammal.

In another preferred embodiment, the ear chondrocytes are derived from autologous ear chondrocytes or xenogenous ear chondrocytes, preferably autologous ear chondrocytes.

In another preferred embodiment, the ear chondrocytes are taken from the subject's autologous ear chondrocytes.

In another preferred embodiment, the subject is a human or a non-human mammal.

In another preferred embodiment, the subject has a joint defect.

In another preferred embodiment, the joint defect is an articular cartilage defect.

In another preferred embodiment, the joint defect is a knee joint defect, an elbow joint defect, a hip joint defect, an ankle joint defect, a wrist joint defect, a mandibular joint defect, and a combination thereof.

In another preferred embodiment, the ear cartilage sheet pieces comprise a cell population composed of chondrocytes and extracellular matrix secreted by chondrocytes, wherein the extracellular matrix encapsulates the cell population, and the ear cartilage pieces are prepared by mincing the ear cartilage sheet, wherein the density of ear chondrocytes is at least 1.0×10⁸ cells/ml or 1.0×10⁸ cells/g.

In another preferred embodiment, the concentration of chondrocytes in the ear cartilage sheet is 1.0×10⁸ cells/ml-10×10⁸ cells/ml, preferably 1.5-5×10⁸ cells/ml.

In another preferred embodiment, the ear cartilage sheet is obtained by gelation culture for 6-30 days, preferably 7-20 days, and most preferably 10-15 days.

In another preferred embodiment, the gelation culture is an in vitro culture with gelation medium.

In another preferred embodiment, the gelation medium contains the following components: high-glucose DMEM medium containing 4 to 5 wt % glucose, 10% FBS (v/v) and 100 U/ml penicillin-streptomycin.

In another preferred embodiment, the thickness of the ear cartilage sheet is 0.2-0.25 mm.

In another preferred embodiment, the average volume of the ear cartilage sheet pieces is 0.2 μl.

In another preferred embodiment, the surface area of the ear cartilage sheet pieces is 0.05-10 mm², preferably, 1-5 mm², more preferably, the average area is 1 mm².

In another preferred embodiment, the porous frame structure is made of a biodegradable material selected from the group consisting of PCL, PGA, allogeneic bone repair material, xenogeneic bone repair material, or decalcified bone matrix.

In another preferred embodiment, the frame structure may further be loaded with gelatin, collagen, silk fibroin, hydrogel, and a combination thereof.

In another preferred embodiment, the frame structure is a PCL frame.

In another preferred embodiment, the frame structure is a decalcified bone matrix.

In another preferred embodiment, the frame structure is an allogeneic bone repair material.

In another preferred embodiment, the allogeneic bone repair material is a decalcified bone matrix.

In another preferred embodiment, the frame structure is a xenogeneic bone repair material.

In another preferred embodiment, the xenogeneic bone repair material is a decalcified bone matrix.

In another preferred embodiment, the shape of the frame structure comprises a cylinder, a cuboid or other specific shape.

In another preferred embodiment, the ear cartilage gel or ear cartilage sheet pieces-frame structure complex can be converted into articular cartilage under the joint microenvironment.

The second aspect of the present invention provides a method for preparing the ear cartilage tissue engineering complex according to the first aspect of the present invention, comprising the steps of: inoculating the ear cartilage gel or ear cartilage sheet pieces according to the first aspect of the present invention into a porous frame structure, and performing in vitro chondrogenic culture, thereby obtaining the ear cartilage tissue engineering complex.

In another preferred embodiment, the ear cartilage gel is inoculated into the porous frame structure by a direct filling method.

In another preferred embodiment, the ear cartilage sheet pieces are inoculated into the porous frame structure by method of centrifugation.

In another preferred embodiment, no liquid is added to the centrifugal system of the method of centrifugation, and the ear cartilage sheet pieces are allowed to enter the frame structure by repeated centrifugation.

In another preferred embodiment, the chondrogenic culture is an in vitro culture using a chondrogenic medium.

In another preferred embodiment, the chondrogenic medium has the following components: high-glucose DMEM medium, serum substitute, proline, vitamin C, transforming growth factor-β1 (TGF-β1), insulin-like growth factor 1 (IGF-I) and dexamethasone.

In another preferred embodiment, the serum substitute is ITS premix, which comprises insulin, transferrin, selenite, linoleic acid, bovine serum albumin, pyruvate, ascorbic acid phosphate.

In another preferred embodiment, the time for chondrogenic culture is 3-15 days, preferably 5-11 days.

The third aspect of the present invention provides a use of the ear cartilage tissue engineering complex according to the first aspect of the present invention for preparing a medical product for repairing joint defects.

In another preferred embodiment, the joint defect is an articular cartilage defect.

In another preferred embodiment, the joint defect is a knee joint defect, an elbow joint defect, a hip joint defect, an ankle joint defect, a wrist joint defect, a mandibular joint defect, and a combination thereof.

The fourth aspect of the present invention provides a method for repairing joint defects, wherein the ear cartilage tissue engineering complex according to the first aspect of the present invention is used to transplant into the defective joint of a patient to be repaired.

In another preferred embodiment, the joint defect is an articular cartilage defect.

In another preferred embodiment, the joint defect is a knee joint defect, an elbow joint defect, a hip joint defect, an ankle joint defect, a wrist joint defect, a mandibular joint defect, and a combination thereof.

It should be understood that within the scope of the present invention, each technical features of the present invention described above and in the following (such as examples) may be combined with each other to form a new or preferred technical solution, which is not listed here due to space limitations.

### DESCRIPTION OF THE DRAWINGS

FIG. 1 shows the structural characteristics of natural elastic cartilage and hyaline cartilage. The results of HE staining, Saf-O staining, COL-II staining and Elastin staining of the two kinds of cartilage are shown respectively.

FIG. 2 shows the results of the ear chondrocyte gel complex and the articular chondrocyte gel complex after 6 months of implantation in the articular cartilage. A and D are integral articular cartilage, in which the implanted regenerated cartilage tissue is circled with dotted lines and displayed in B and E; C and F are the sectional views of the implanted regenerated cartilage tissue.

FIG. 3 shows the effect of the articular cartilage microenvironment on the types of ectopic chondrogenesis of ear chondrocytes and articular chondrocytes. The results of HE staining, Saf-O staining, COL-II staining and Elastin staining of two kinds of regenerated cartilage are shown respectively. The bar in the figure is 150 μM.

FIG. 4 shows a general schematic of the decalcified bone matrix frame. The bar in the figure is 1 cm.

FIG. 5 shows the electron microscope of the decalcified bone matrix frame, and the bar in the figure is 1 mm.

FIG. 6 shows schematic diagrams of ear cartilage gel and ear cartilage sheet obtained by culturing ear chondrocytes for 3 days and 15 days. Among them, A-C show ear cartilage gel cultured for 3 days, D-E show ear cartilage sheet cultured for 15 days (D and E), and ear cartilage sheet pieces formed by mincing (F).

FIG. 7 shows a physical diagram of the ear cartilage gel-frame structure complex. The bar in the figure is 1 cm.

FIG. 8 shows a physical diagram of the ear cartilage sheet pieces-frame structure complex. The bar in the figure is 1 cm.

FIG. 9 shows a comparison of the adhesion rate of the inoculated sample (cell suspension or cartilage gel) after culture on the decalcified bone matrix for 24 hours.

FIG. 10 shows a comparison of gel cartilage-decalcified bone complex (A) and simple decalcified bone (B) transplanted to the knee joint defect site in a goat.

### DETAILED DESCRIPTION

After extensive and in-depth research, the inventors surprisingly discovered and developed an ear cartilage tissue engineering complex for the first time. The ear cartilage tissue engineering complex is an integrated ear cartilage gel/ear cartilage sheet pieces-frame structure complex. Experiments have proved that by obtaining the primary ear cartilage for expansion culture, a specific number of ear chondrocytes are inoculated on and/or spread on a flat or basically flat culture surface, so that the inoculated chondrocytes can form a specific laminated structure, and the laminated chondrocytes are cultured under suitable gelation culture conditions. A novel gel-like ear cartilage or sheet-like ear cartilage can be formed due to different culture times. Combining the prepared gel-like cartilage or sheet-like cartilage with the porous frame structure, an ear cartilage tissue engineering complex can be prepared. The prepared ear cartilage tissue engineering complex can be transformed into articular cartilage at the defective joint after being transplanted into the defective joint, so that the articular cartilage can be repaired and reconstructed. On this basis, the present invention has been completed.

### Term

Unless otherwise defined, all technical and scientific terms used herein have the same meanings as those commonly understood by those skilled in the art to which the present invention belongs.

As used herein, the term “ear cartilage tissue engineering complex” includes the ear cartilage gel-frame complex and the ear cartilage sheet pieces-frame complex that have been chondrogenic cultured in vitro or have not been chondrogenic cultured in vitro as described herein. In the present invention, they can be uniformly referred to as ear cartilage tissue engineering complex.

As used herein, the term “inoculation” means the inoculation of ear chondrocytes in a cell culture dish, and may also mean the inoculation of ear cartilage gel/ear cartilage sheet pieces into a specified frame structure and the uniform distribution thereof. The meaning of “inoculation” as used can be understood by those skilled in the art from the context.

As used herein, when used in reference to specific enumerated values, the term “about” means that the value may vary by no more than 1% from the enumerated value. For example, as used herein, “about 100” includes all values between 99 and 101 (e.g., 99.1, 99.2, 99.3, 99.4, etc.).

As used herein, the term “contains” or “includes (comprises)” can be open, semi-closed and closed. In other words, the term also includes “substantially consisting of . . . ” or “consisting of . . . ”.

### Ear Cartilage Gel and Preparation Thereof

As used herein, “gel ear cartilage”, “ear cartilage gel”, “gel-state ear cartilage”, “gel-like ear cartilage”, “ear cartilage gel of the present invention” or “gel ear cartilage of the present invention” can be used interchangeably, all refer to the ear cartilage (stem) cells in gel state of the present invention, in particular, ear chondrocytes with a specific concentration are inoculated on and/or spread on a flat or substantially flat culture surface, so that the inoculated ear chondrocytes form a laminated structure, and the ear chondrocytes having a laminated structure are cultured under suitable gelation culture conditions, thereby forming a gel-like ear cartilage culture.

The gel ear cartilage of the present invention is a new type of ear cartilage different from free ear chondrocytes, centrifugally precipitated ear chondrocytes and ear cartilage pellet. The gel ear cartilage of the present invention can be regarded as a specific form of ear cartilage between free ear chondrocytes and dense ear cartilage pellet. During the process of gelation culture of the gel ear cartilage of the present invention, the ear chondrocytes not only contact and/or interact with adjacent cells on the plane (X-Y plane), but also contact and/or interact with adjacent chondrocytes in multiple directions such as above and/or below and/or the upper or lower side, so as to promote the ear chondrocytes to secrete and form more extracellular matrix, therefore, the gelation cultured ear chondrocytes are wrapped in extracellular matrix with a certain viscosity, so that the gel ear cartilage of the present invention has a close connection, but has a certain viscosity and fluidity, so that the gel ear cartilage of the present invention is more suitable for inoculation and loading on various carrier materials (especially porous carrier materials), thereby forming a complex for repairing ear cartilage.

In addition, the gel ear cartilage of the present invention has a gel state on the one hand, and on the other hand, it has an unusually high cell density (usually at least 1.0×10⁸ cells/ml or more, such as 1.0×10⁸-10×10⁸ cells/ml). Therefore, it is particularly suitable for the preparation of grafts for repairing various types of ear cartilage, or for ear cartilage transplantation or ear cartilage repair surgery.

In the present invention, the complex for repairing ear cartilage includes the complex formed by loading the gel ear cartilage of the present invention on a carrier material (especially a porous biological frame structure) without undergoing chondrogenic culture, and also includes the complex formed by loading the gel ear cartilage of the present invention on a carrier material (especially a porous biological frame structure) and undergoing chondrogenic culture.

In the present invention, the complex suitable for transplantation to a human or animal body is the ear cartilage tissue engineering complex of the present invention, that is, the complex formed by loading the gel ear cartilage of the present invention on a carrier material (especially a porous biological frame structure) and undergoing chondrogenic culture.

Preferably, in the present invention, the gel ear cartilage is formed by in vitro culture for a period of time t1 under the gelation culture condition. Preferably, the t1 is 2.5-5.5 days, preferably 3-5 days.

In the present invention, one feature is laminated inoculation, that is, after ear chondrocytes with a specific density are inoculated into a culture container, the inoculated ear chondrocytes will form a multilayer ear chondrocyte group (i.e., an ear chondrocyte group with a laminated structure) through, for example, deposition. Typically, calculated on the basis of the culture area of the culture dish (or culture container), and assuming that the degree of confluence of the monolayer cells is 100%, the cell number S1 of the laminated inoculation of the present invention is n times of the cell number S0 for the of 100% confluence degree (i.e., S1/S0=n), wherein n is 1.5-20, preferably 2-10, more preferably 2.5-5.

### Ear Cartilage Sheet and Preparation Thereof

As used herein, “ear cartilage sheet”, “sheet-like ear cartilage”, or “ear cartilage sheet of the present invention” can be used interchangeably, all refer to the ear cartilage (stem) cells in sheet state of the present invention, in particular, referring to the ear chondrocytes with a specific concentration are inoculated on and/or spread on a flat or substantially flat culture surface, so that the inoculated ear chondrocytes form a laminated structure, and the ear chondrocytes having a laminated structure are cultured under suitable culture conditions, thereby forming a sheet-like ear cartilage culture.

The “ear cartilage sheet” of the present invention is prepared on the basis of the preparation of the “ear cartilage gel” of the present invention by prolonging the gelation culture time. That is, in the present invention, the ear chondrocytes are inoculated and/or spread on a flat or substantially flat culture surface and cultured in vitro for a period of time t2 under gelation culture conditions, thereby forming an ear cartilage sheet. Preferably, the t2 is 6-30 days, preferably 7-20 days, and most preferably 10-15 days.

On the one hand, the ear cartilage sheet of the present invention has an unusually high cell density (usually at least 1.0×10⁸ cells/ml or more, such as 1.0×10⁸-10×10⁸ cells/ml). On the other hand, its thickness is thin (only 0.2-0.25 mm) and it has good toughness, and can be cut into “ear cartilage sheet pieces” with an average volume of 0.2 μl. They can be filled in the porous frame structure by simple centrifugation. Therefore, it is especially suitable for preparing and repairing various types of cartilage grafts, or for cartilage transplantation or cartilage repair surgery.

In the present invention, the complex for repairing ear cartilage includes the complex formed by loading the ear cartilage sheet pieces of the present invention on a carrier material (especially a porous frame structure) without undergoing chondrogenic culture, and also includes the complex formed by loading the ear cartilage sheet pieces of the present invention on a carrier material (especially a porous frame structure) and undergoing chondrogenic culture.

In the present invention, the complex suitable for transplantation to a human or animal body is the ear cartilage tissue engineering complex of the present invention, that is, the complex formed by loading the ear cartilage sheet pieces of the present invention on a carrier material (especially a porous frame structure) and undergoing chondrogenic culture.

As used herein, “specific concentration” or “specific density” refers to inoculating 1.0×10⁷-2.0×10⁷ cells, preferably 1.5×10⁷ cells, into a 3.5 cm culture dish (e.g., one well in a six-well plate). After gelation culture for different times, the ear cartilage gel containing ear chondrocytes with a density of 1.0×10⁸-10×10⁸ cells/ml or the ear cartilage sheet containing ear chondrocytes with a density of 1.0×10⁸-10×10⁸ cells/ml is finally formed.

In another preferred embodiment, the gelation culture condition is: inoculating chondrocytes with a specific density, and using gelation medium for culture, wherein the gelation medium is high-glucose (4-5 wt % glucose) DMEM medium containing 10% fetal bovine serum and 100 U/ml penicillin-streptomycin.

As used herein, the term “chondrogenic culture” refers to culturing a porous frame structure inoculated with ear cartilage gel or ear cartilage sheet pieces using chondrogenic medium to eventually form an integrated ear cartilage gel-frame structure complex or ear cartilage sheet pieces-frame structure complex, i.e., the ear cartilage tissue engineering complex of the present invention, for transplantation into cartilage defects in human or animal bodies.

### Cartilage and Chondrocytes

Cartilage refers to cartilage tissue, which is composed of chondrocytes and intercellular substance. The matrix in the cartilage is in gel state and has greater toughness. Cartilage is a connective tissue with main supporting function. There are no blood vessels and lymphatic vessels in cartilage. Nutrients infiltrate into the intercellular substance from the blood vessels in the perichondrium and then nourish bone cells.

Cartilage can be divided into 3 types according to the different intercellular substance, namely hyaline cartilage, elastic cartilage and fibrocartilage. The matrix of hyaline cartilage is composed of collagen fibers, fibrils and surrounding amorphous matrix. During the embryonic period, it has the function of temporary scaffold, which is later replaced by bone. In adults, hyaline cartilage is mainly distributed in the trachea and bronchial wall, the sternal end of the ribs and the surface of the bone (articular cartilage). In addition to collagen fibers, there are elastic fibers in the matrix of elastic cartilage. This kind of cartilage has greater elasticity and is mainly distributed in the auricle, external auditory canal wall, eustachian tube, epiglottis, and throat, etc. There are bundles of collagen fibers in the fibrocartilage matrix arranged in parallel or cross, which are tough. It is distributed in the intervertebral disc, glenoid, articular disc and some tendons, ligaments, to enhance the flexibility of movement and protection, support and other functions.

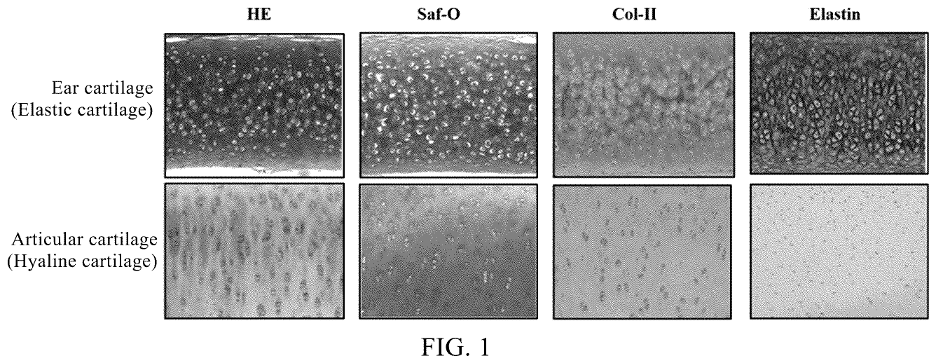

The biggest difference between hyaline cartilage and elastic cartilage is that the former (i.e., hyaline cartilage) expresses rich elastin. The structural characteristics of the both are shown in FIG. 1.

In a preferred embodiment of the present invention, autologous ear chondrocytes (elastic cartilage) taken from a subject with joint defects are cultured in vitro to prepare ear cartilage gel or ear cartilage sheet pieces, and the ear cartilage gel or ear cartilage sheet pieces are inoculated on the porous frame structure to prepare the ear cartilage gel-frame complex or the ear cartilage sheet pieces-frame complex, which can be used for articular cartilage (hyaline cartilage) repair of joint defects.

### HE Staining, Saf-O Staining, COL-II Staining and Elastin Staining

HE staining: hematoxylin-eosin staining, referred to as HE staining, is one of the commonly used staining methods in paraffin sectioning technology. The hematoxylin dye solution is alkaline, which mainly makes the chromatin in the nucleus and the nucleic acid in the cytoplasm purple-blue; the eosin is an acid dye, which mainly makes the components in the cytoplasm and extracellular matrix red.

Saf-O staining: also known as safranine O staining, is a commonly used cartilage staining method. The principle of Saf-O staining is that the basophilic cartilage combines the basic dye safranine O to present red. Safranine O is a cationic dye that combines multiple anions, which shows that cartilage tissue combines polysaccharide anionic groups (chondroitin sulfate or keratin sulfate) based on cationic dyes.

COL-II staining: COL-II is type II collagen, which is a kind of high molecular protein. Filamentous collagen fibers interweave with elastin and polysaccharide to form a network structure, resulting in certain mechanical strength. Type II collagen is mainly produced by chondrocytes and mostly exists in bones, joints, tendons and other tissues.

Elastin staining: Elastin is elastin protein, which is the main component of elastic fibers. There are two forms of elastin have been found: elastin I is found in the ligaments, aorta and skin, and elastin II can be obtained from cartilage. In examples of the present invention, Elastin staining is used to reflect the expression of elastin in chondrocytes to distinguish different types of chondrocytes, such as ear cartilage and articular cartilage.

### Cartilage Transformation

As used herein, the term “cartilage transformation” refers to the transformation between different types of cartilage, i.e., the transformation from one type of cartilage to another type of cartilage, or the transformation from one type of chondrocytes to another type of chondrocytes.

In the present invention, the ear chondrocytes (hyaline chondrocytes) in the ear cartilage tissue engineering graft formed by in vitro culture are transformed into articular chondrocytes (elastic chondrocytes) under the joint tissue microenvironment after being transplanted into the defective joint, and finally form articular cartilage.

### MACI and ACI

MACI is the abbreviation of matrix-induced autologous chondrocyte implantation, which means “matrix-induced autologous chondrocyte transplantation”. It is a technology that uses tissue engineering technology to carry out chondrocyte transplantation. MACI is the latest and best technology for the treatment of articular cartilage defects in the world.

ACI is the abbreviation of autologous chondrocyte implantation, which means “autologous chondrocyte transplantation”. It is one of the widely used tissue engineering techniques for the treatment of articular cartilage defects. After the advent of MACI, ACI was called “traditional ACI” accordingly to distinguish it from MACI.

### Porous Frame Structure

As used herein, the term “porous frame structure” refers to a carrier made of a biocompatible material having a certain number of pores on its surface and interior to facilitate the attachment of the ear cartilage gel or ear cartilage sheet inoculated thereon. In the present invention, the biocompatible material is preferably a biodegradable material.

A biodegradable material refers to a material that can be decomposed in the body after being transplanted into an animal. The porous frame structure of the ear cartilage tissue engineering graft of the present invention is made of a biodegradable material selected from the group consisting of PCL, PGA, allogeneic bone repair material, xenogeneic bone repair material, decalcified bone matrix, and a combination thereof, but not limited to the materials described above. Among them, allogeneic bone repair materials and xenogeneic bone repair materials include decalcified bone matrix materials. In a preferred embodiment of the present invention, the porous frame structure of the ear cartilage tissue engineering complex is a decalcified bone matrix.

### Decalcified Bone Matrix

The decalcified bone matrix used in the preferred embodiment of the present invention has a thickness of 0.3-0.8 cm, preferably 0.4-0.6 cm, and most preferably 0.5 cm. The decalcification amount of the decalcified bone matrix is 30% to 50%, the degree of decalcification of which is appropriate, and the supporting effect is good. and the decalcified bone matrix is easy to trim and cut to a suitable shape and size. The pore size of the demineralized bone matrix is 400-800 μm, which is easy to be filled with chondrocytes.

Decalcified bone matrix (DBM) is a bone graft material that can reduce immunogenicity by decalcification of allogeneic bone or xenogeneic bone. Different degrees of decalcification correspond to different mechanical strengths. It has good biological characteristics, osteoinductivity, osteoconductivity and biodegradability, which promotes new bone formation and bone tissue mineralization, and then accelerates bone healing. It can effectively repair bone damage alone or in combination with autologous bone, other biological materials and growth factors. It is an ideal bone tissue engineering scaffold material. However, the general decalcified bone matrix has a large pore size and extremely low cell adhesion rate when inoculated with chondrocyte suspension, which is not conducive to the construction of tissue engineering carriers.

In another preferred embodiment, the decalcified bone matrix of the present invention has a pore size of 400-800 μm and a porosity of 87.3%±3.7%.

### PCL

Polycaprolactone (PCL) is a fully biodegradable polymer material. It is formed by ring-opening polymerization of ε-caprolactone under the conditions that metal organic compounds (such as tetrapylethyltin) are used as catalyst, and dihydroxy or trihydroxy are used as initiator. It belongs to polymerized polyester, and its molecular weight and degree of disproportionation vary with the type and amount of starting material. Its appearance is white solid powder, non-toxic, insoluble in water, and easily soluble in various polar organic solvents. PCL has good biocompatibility, good organic polymer compatibility, and good biodegradability. It can be used as a cell growth support material and can be compatible with a variety of conventional plastics. It can be completely degraded in a natural environment within 6-12 months. In addition, PCL also has good shape memory temperature control properties, and is widely used in the production and processing of drug carriers, plasticizers, degradable plastics, nanofiber spinning, and molding materials.

In a preferred embodiment, the ear cartilage gel/ear cartilage sheet pieces of the present invention can be inoculated on the PCL frame structure to form an ear cartilage gel-frame complex or an ear cartilage sheet pieces-frame complex for repairing articular cartilage.

### Allogeneic Bone Repair Material

Allogeneic bone is currently the most commonly used bone implantation material in orthopedics. It is mainly used to repair and fill bone defects and play a role of fixation and support. Allogeneic bone is taken from donated human bone tissue. “Allogeneic” indicates that it is from the human body but not from the patient's own body. After the donor is selected, it is usually obtained under sterile conditions within 24 hours of death, and processed immediately. Preservation methods include fresh freezing and freeze drying. Fresh frozen bone can be stored for 1 year at −20° C.; freeze dried bone can be stored at room temperature for a long time after vacuum packaging, and the antigenicity of that is lower. Compared with fresh frozen bone, the mechanical properties of freeze dried bone will be reduced by 50%, and ethylene oxide or high-dose γ-ray irradiation disinfection will further reduce the bone induction performance.

In a preferred embodiment, the ear cartilage gel/ear cartilage sheet pieces of the present invention can be inoculated on a frame structure (e.g., decalcified bone matrix) prepared by allogeneic bone to form an ear cartilage gel-frame complex or ear cartilage sheet pieces-frame complex for repairing articular cartilage.

### Xenogenic Bone Repair Material

Xenogenic bone is a bone repair material derived from other species such as cattle or pig, etc. It has a wide range of sources and relatively low prices. However, the immunogenicity of xenogeneic bone is strong, and it is easy to cause immune rejection after implantation in patients. In addition, xenogeneic bone has no ability to induce the proliferation of mesenchymal stem cells, and its biological activity is poor. It needs to be combined with other repair materials or related cytokines to achieve the repair effect.

In a preferred embodiment, the ear cartilage gel/ear cartilage sheet pieces of the present invention can be inoculated on a frame structure (e.g., decalcified bone matrix) prepared by xenogeneic bone to form an ear cartilage gel-frame complex or ear cartilage sheet pieces-frame complex for repairing articular cartilage.

### The Medium Used in the Present Invention

Chondrogenic medium: high glucose DMEM medium, 1% 1×ITS premium (ITS universal culture mixture, containing insulin, transferrin, selenite, linoleic acid, bovine serum protein, pyruvate, ascorbic acid phosphate), 40 μg/ml proline, 10 ng/ml TGF-β 1, 100 ng/ml IGF-1, 40 ng/ml dexamethasone and 50 μg/ml vitamin C.

Gelation medium: DMEM medium containing 4-5 wt % glucose, 10% FBS (v/v) and 100 U/ml penicillin-streptomycin.

### Adhesion Rate

In the present invention, when the ear cartilage gel of the present invention is inoculated into a carrier material (especially a porous biological frame structure), the ear cartilage gel of the present invention has a certain adhesion rate, which is determined by the adhesion rate measurement method provided by the present invention. The adhesion rate of the ear cartilage gel of the present invention is ≥90%, preferably ≥95%.

The adhesion rate in the present invention is defined as follows:

Detect the DNA quantification A1 of inoculated samples (e.g., ear cartilage gel); Detect the DNA quantification A2 of the post-inoculation complex (e.g., ear cartilage gel-frame complex) after culture for 24 hours. The adhesion rate is A2/A1*100%.

The method of determining the adhesion rate includes the following steps:

Taking inoculated samples (such as ear cartilage gel or ear cartilage gel-frame complex) and digesting them with protease K. The digested samples are quantitatively detected using PicoGreen kits (Invitrogen, Carlsbad, CA, USA), absorbance of 520 nm is determined using fluorescent microplate reader, and DNA content is calculated according to standard curve formula.

### The Main Advantages of the Present Invention Include

(1) Ear cartilage gel is more mature than chondrocytes and has certain fluidity.
(2) The source of cells is ear cartilage, which does not need to be taken from the joint site, and it will not cause secondary damage to the patient's joint area, and ear chondrocytes will be transformed into articular chondrocytes in the joint environment.
(3) The expansion ability of ear cartilage is significantly better than that of articular cartilage, and the waiting period of patients is short.
(4) Most of the selected frame structures are natural materials or materials with neutral degradation products. The frame structures have moderate degradation rate in vivo, low immune response and good biological safety.
(5) The frame structure material has a large pore size and good porosity, but the cell adhesion rate is extremely low when inoculated with chondrocyte suspension. The use of cartilage gel-like tissue with certain fluidity and viscosity can effectively improve the adhesion rate.
(6) Compared with other tissue engineering repair methods, ear cartilage gel/ear cartilage sheet pieces-frame structure complex can regenerate cartilage stably.
(7) Ear cartilage gel/ear cartilage sheet pieces-frame structure complex can regenerate cartilage stably and provide real-time mechanical support. The real-time repair effect of that is good.

Hereinafter, the present invention will be further described with reference to specific examples. The present invention is further explained below in conjunction with specific example. It should be understood that these examples are only for illustrating the present invention and not intend to limit the scope of the present invention. The conditions of the experimental methods not specifically indicated in the following examples are usually in accordance with conventional conditions as described in Sambrook et al., Molecular Cloning: A Laboratory Manual (New York: Cold Spring Harbor Laboratory Press, 1989), or according to the conditions recommended by the manufacturer. Unless otherwise stated, percentages and parts are calculated by weight. Unless otherwise stated, the materials and reagents used in the examples of the present invention are commercially available products.

### Example 1

### Study on the Outcome of Different Types of Cartilage in Natural Articular Cartilage Microenvironment

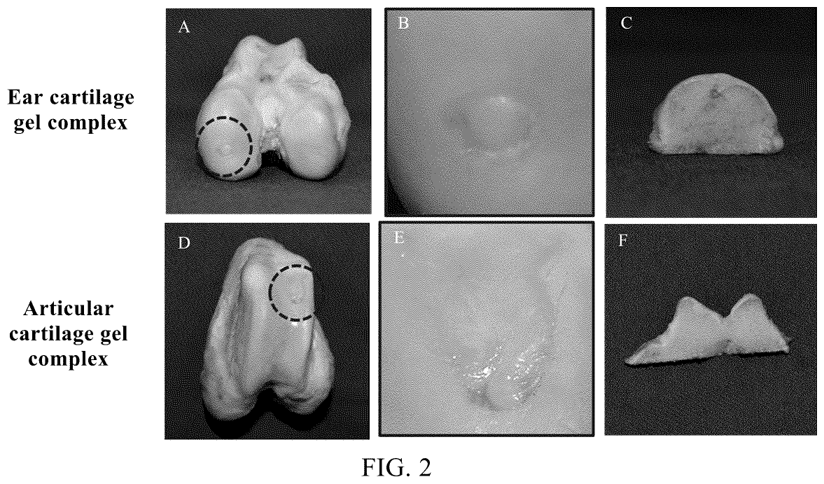

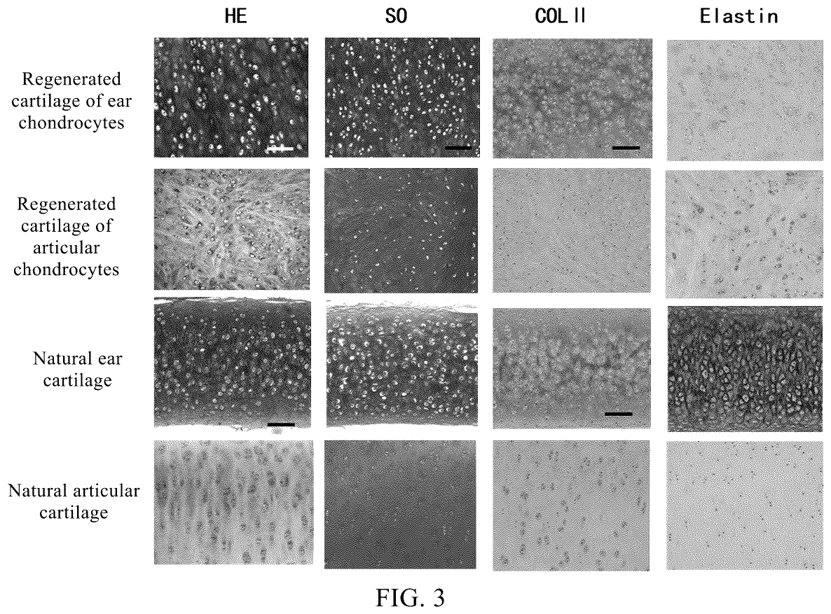

According to the different structure of the extracellular matrix, cartilage tissue is divided into three types: elastic cartilage, hyaline cartilage, and fibrocartilage. Among them, ear cartilage is elastic cartilage and articular cartilage is hyaline cartilage. The biggest difference between the two is that the former expresses abundant elastin (see FIG. 1). In order to study the outcome of different types of cartilage in the natural articular cartilage microenvironment, the following experiments were carried out.
(1) Separation and culture of two kinds of chondrocytes: after the goat was anesthetized, a part of the auricle was cut under sterile conditions, and the cartilage in the non-weight-bearing area of the joint was scraped at the same time. The cartilage was cut into 1 mm×1 mm pieces under sterile conditions. 0.15% collagenase was prepared, and the cartilage pieces were added to the prepared collagenase for digestion for 8 hours. After 8 hours, the collagenase solution was filtered and centrifuged to obtain ear chondrocytes. Primary culture and subculture were conducted to expand the cells to a required amount.
(2) Preparation of Pluronic temperature-sensitive gel: pluronic powder and H-DMEM were mixed at room temperature in a ratio of 3:10, then stirred overnight in a refrigerator at 4° C., and finally sterilized at high temperature for later use.
(3) Preparation of cell gel mixture: 0.25% trypsin was used to digest and collect cells respectively. The cells were counted, and 100×10⁶ cells were taken and fully mixed with Pluronic temperature-sensitive gel. Placed in the incubator for 30 min, and washed twice with PBS to discard the supernatant. The cells precipitate was pre-cooled in an ice box, and 1 ml of gel was added to every 100×10⁶ cells and fully mixed. The cell gel mixture was extracted with a 1 ml syringe fixed with a 20 ml needle. With the syringe in hand, the gel quickly became semi-solid to keep the cells evenly distributed in the gel.
(4) Three kinds of chondrocytes-gel complex were implanted back into the articular cartilage microenvironment: after the goat was anesthetized. The bare joints of the surgical side were wrapped with sterile towels. The knee joint was extended and bent to determine the position of patellar activity. A longitudinal arc incision of about 8-10 cm was made along the posterior edge of the patellar ligament. The upper part reached about 2-3 cm above the upper edge of the patella, and the lower end reached about 1 cm below the articular tubercle. The skin was lifted inwardly, and the subcutaneous, superficial fascia and deep fascia were cut in layers with large blood vessels and meningeal scissors. Open the joint capsule and synovium along the posterior edge of the primary zone, and pay attention not to damage the primary zone. The trochlea was fully exposed, and a circular defect of full-thickness cartilage was created with a 5 mm diameter trephine. Different chondrocyte gel mixtures were injected into the defect for in vivo culture for six months.
(5) Results of sampling: After six months of in vivo culture, it was found that both ear chondrocyte gel complex and articular chondrocyte gel complex formed translucent cartilage tissue in the articular cartilage microenvironment (see A-C, D-F of FIG. 2), and neither of them expressed elastin (see FIG. 3).

### Example 2

### Preparation of Ear Cartilage Gel-Frame Structure Complex

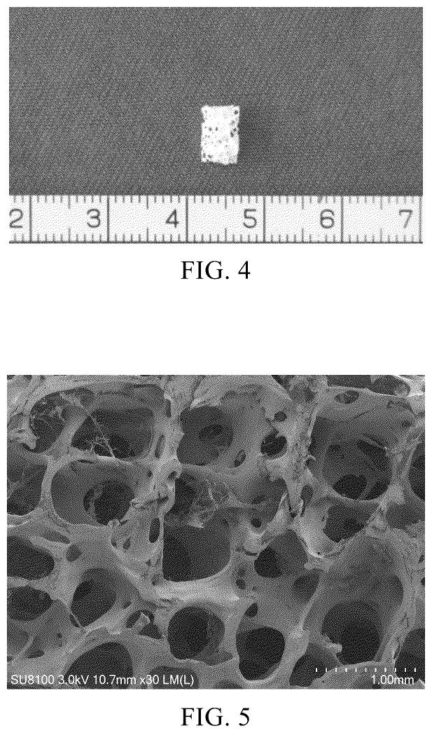

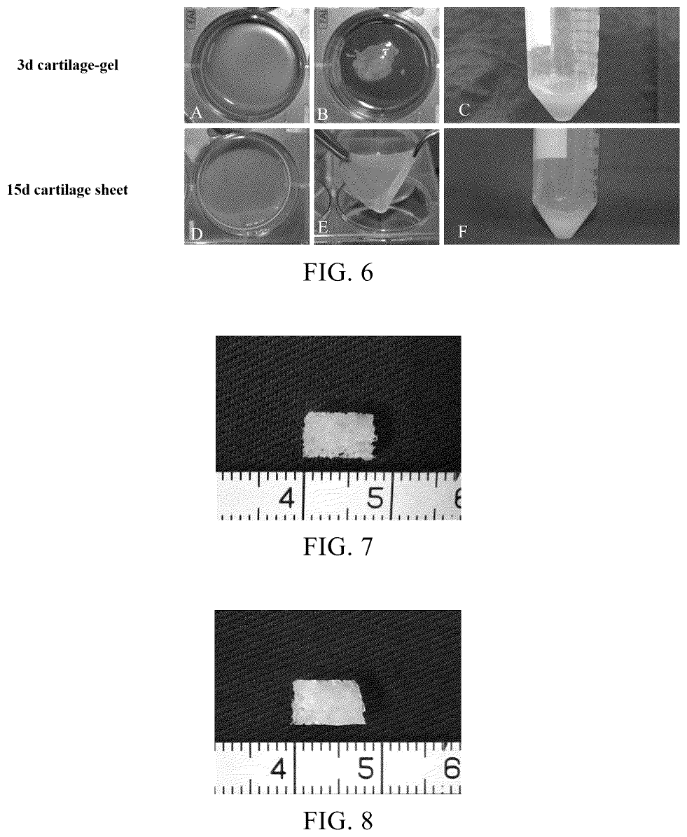

In this example, the ear cartilage gel-frame complex structure was prepared. The specific operation method is as follows:
(1) Part of the subject's autologous ear cartilage tissue was taken, and 2.5×2.5 cm² ear cartilage tissue was aseptically cut. The mucosa and fibrous tissue was peeled off from the cartilage surface by sterile instruments.
(2) The ear cartilage tissue was cut into 1.5×1.5 mm² cartilage pieces; 0.15% collagenase was prepared; and the cartilage pieces were added to the prepared collagenase for digestion for 8 hours.
(3) After 8 hours, collagenase solution was filtered and centrifuged to obtain ear chondrocytes, and primary and subculture were conducted using high-glucose DMEM medium containing 10% FBS;
(4) After expansion, the cells were collected and resuspended, and the cells were inoculated into a six-well plate with the cell volume of 8×10⁶ cells/10 ml to 30×10⁶ cells/10 ml/well, and were cultured in gelation medium (DMEM medium containing 4-5 wt % glucose, 10% FBS (v/v) and 100 U/ml penicillin-streptomycin).
(5) After 72 hours (3 days) of inoculation, the medium in the six-well plate was sucked off, and the gel-like ear cartilage tissue at the bottom of the six-well plate can be seen (A of FIG. 6). The gel-like ear cartilage at the bottom of the six-well plate was gathered by using a tweezer (B of FIG. 6). The gel cartilage yield of one well was 0.1-0.2 ml and collected into a 5 ml syringe. In the gel cartilage, the cell density was 1.0×10⁸ cells/ml-10×10⁸ cells/ml, preferably 1.5-5×10¹⁰ cells/ml. It was mixed with 0.15 ml of medium to make a preparation containing ear cartilage gel, as shown in C of FIG. 6.
(6) The ear cartilage gel preparation (prepared in step (5), volume was about 0.25-0.35 ml) was inoculated in the frame structure (decalcified bone matrix frame (FIG. 4 and FIG. 5, with a pore size of about 400-800 μm, and a porosity of about 87.3%±3.7%), and placed at 37° C., 95% humidity, 5% CO₂ for 2 hours.
(7) Then chondrogenic medium was added to continue in vitro culture for 3-11 days to form the ear cartilage gel-frame structure complex, as shown in FIG. 7.

When used for joint defect repair, the shape and size of the cartilage to be repaired can be determined according to the previous MRI, CT and other auxiliary examinations to cut the ear cartilage gel-frame structure complex.

### Example 3

### Preparation of Ear Cartilage Sheet Pieces-Frame Structure Complex

In this example, the ear cartilage sheet pieces-frame complex was prepared. The specific operation method is as follows:
(1) 2.5×2.5 cm² ear cartilage tissue was aseptically cut, and the mucosa and fibrous tissue was peeled off from the cartilage surface by sterile instruments.
(2) The ear cartilage tissue was cut into 1.5×1.5 mm² cartilage pieces; 0.15% collagenase was prepared; and the cartilage pieces were added to the prepared collagenase for digestion for 8 hours.
(3) After 8 hours, collagenase solution was filtered and centrifuged to obtain ear chondrocytes for primary and subculture.
(4) After expansion, the cells were collected and resuspended, and the cells were inoculated into a six-well plate with the cell volume of 8×10⁶ cells/10 ml to 30×10⁶ cells/10 ml/well, and cultured in gelation medium (DMEM medium containing 4-5 wt % glucose, 10% FBS (v/v) and 100 U/ml penicillin-streptomycin). After 24 hours or 48 hours of culture, the fresh gelation culture medium was replaced and the cells were cultured in vitro until Day 15.
(6) The culture medium in the six-well plate was removed, and the ear cartilage sheet tissue at the bottom of the six-well plate was visible (D of FIG. 6), in which the cell density in the ear cartilage sheet tissue was about 1.0×10⁸ cells/ml-10×10⁸ cells/ml.

The ear cartilage sheet was clamped with tweezers (E of FIG. 6), which was cut into 1×1 mm² ear cartilage sheet pieces, and then collected into a 50 ml centrifuge tube, as shown in F of FIG. 6.
(7) The frame material to be inoculated (decalcified bone matrix frame (FIG. 4 and FIG. 5)) was placed in a centrifuge tube containing ear cartilage sheet pieces to ensure that the frame material is completely submerged. The centrifuge tube containing the frame material and ear cartilage sheet pieces was placed in a centrifuge at 600 r/min minutes for 2 minutes.
(8) The inoculated frame material was placed at 37° C., 95% humidity, and 5% CO₂ for a certain period of time; then chondrogenic medium was added to continue in vitro culture for 3-11 days to form the ear cartilage sheet pieces-frame structure complex, as shown in FIG. 8.

When used for joint defect repair, the shape and size of the cartilage to be repaired can be determined according to the previous MRI, CT and other auxiliary examinations to cut the ear cartilage sheet pieces-frame structure complex.

### Comparative Example 1

### Detection of Adhesion Rate of Chondrocyte Suspension and Cartilage Gel

A decalcified bone matrix frame was provided (as shown in FIG. 4). The gel cartilage preparation (prepared in Example 2 with volume of about 0.25-0.35 ml) was inoculated into the above decalcified bone matrix frame, and the DNA content of the gel cartilage preparation was determined before inoculation.

The primary cultured chondrocytes were subcultured at 37° C., 95% humidity and 5% CO₂ for 4 times, and the cell culture medium was added to prepare the cell suspension, and the DNA content of the prepared chondrocyte suspension was determined. The cell suspension was inoculated into the above decalcified bone matrix frame.

The inoculated cartilage gel-decalcified bone matrix complex and chondrocyte suspension-decalcified bone matrix complex were placed in an incubator at 37° C., 95% humidity, and 5% CO₂ for 24 hours. The two complexes were sampled separately to determine the DNA content of each.

As shown in FIG. 9, the adhesion rate is calculated by the adhesion rate measurement method described in the specification. Compared with the cell suspension, the adhesion rate of the gel cartilage of the present invention is 92%±2%, which is about 3 times of the adhesion rate of cell suspension.

### Comparative Example 2

### Animal Transplantation Experiment for Repairing Articular Cartilage

A cartilage defect with a diameter of 7.5 mm was manufactured on the articular surface of the knee joint of the experimental animal, and the ear cartilage gel-frame complex (gel cartilage-decalcified bone complex) prepared in Example 1 and the simple decalcified bone matrix were implanted at the defect position A and the defect position B, respectively.

The repair of cartilage defects in animals was observed immediately after implantation.

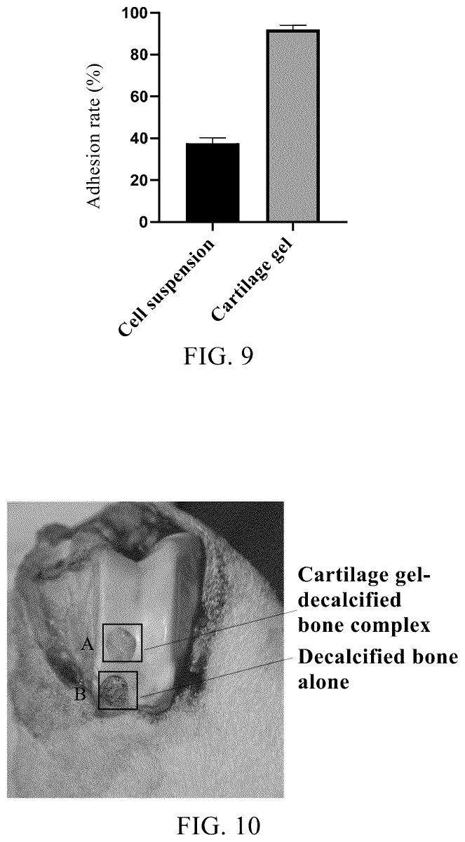

The results are shown in FIG. 10:

The defect at position A is smooth and solid, surrounded by soft tissue membrane, with certain elasticity and excellent immediate repair effect.

The wound surface at the defect position B is rough and has only physical support function, which cannot be used for immediate repair.

All references mentioned in the present application are incorporated by reference herein, as though individually incorporated by reference. In addition, it should be understood that after reading the above teaching content of the present invention, various changes or modifications may be made by those skilled in the art, and these equivalents also fall within the scope as defined by the appended claims of the present application.

1. An ear cartilage tissue engineering complex, which comprises:
(a) a carrier comprising a porous frame structure; and
(b) ear cartilage gel or ear cartilage sheet pieces containing ear chondrocytes that are inoculated on or loaded on the carrier.

2. The complex of claim 1, wherein the ear cartilage gel comprises a cell population of chondrocytes and extracellular matrix secreted by chondrocytes, wherein the extracellular matrix encapsulates the cell population, and the ear cartilage gel is in a gel state, and the density of chondrocytes is at least 1.0×10⁸ cells/ml or 1.0×10⁸ cells/g.

3. The complex of claim 2, wherein the ear cartilage gel is obtained by gelation culture for 2.5-5.5 days, preferably for 3-5 days.

4. The complex of claim 2, wherein the adhesion rate of the ear cartilage gel is≥90%, preferably ≥95%.

5. The complex of claim 1, wherein the ear cartilage sheet pieces comprise a cell population composed of chondrocytes and extracellular matrix secreted by chondrocytes, wherein the extracellular matrix encapsulates the cell population, and the ear cartilage pieces are prepared by mincing the ear cartilage sheet, wherein the density of ear chondrocytes is at least 1.0×10⁸ cells/ml or 1.0×10⁸ cells/g.

6. The complex of claim 5, wherein the ear cartilage sheet is obtained by gelation culture for 6-30 days, preferably 7-20 days, and most preferably 10-15 days.

7. The complex of claim 3, wherein the gelation culture is in vitro culture with a gelation medium containing the following components: high-glucose DMEM medium containing 4 to 5 wt % glucose, 10% FBS (v/v) and 100 U/ml penicillin-streptomycin.

8. The complex of claim 1, wherein the ear chondrocytes are derived from autologous ear chondrocytes or xenogenous ear chondrocytes, preferably autologous ear chondrocytes.

9. The complex of claim 1, wherein the porous frame structure is made of a biodegradable material selected from the group consisting of PCL, PGA, allogeneic bone repair material, xenogeneic bone repair material, or decalcified bone matrix.

10. The complex of claim 1, wherein the ear cartilage gel/ear cartilage sheet pieces-frame structure complex can be converted into articular cartilage under the joint microenvironment.

11. A method for preparing the ear cartilage tissue engineering complex of claim 1, comprising the steps of: inoculating the ear cartilage gel or ear cartilage sheet pieces of into a porous frame structure, and after performing in vitro chondrogenic culture, the ear cartilage tissue engineering complex is obtained.

12. The method of claim 11, wherein the chondrogenic culture is in vitro culture with a chondrogenic medium having the following components: high-glucose DMEM medium, serum substitute, proline, vitamin C, transforming growth factor-β1 (TGF-β1), insulin-like growth factor 1 (IGF-I), and dexamethasone.

13. The method of claim 11, wherein the ear cartilage sheet pieces are inoculated into the porous frame structure by a method of centrifugation, and no liquid is added to the centrifugal system of the centrifugation method, and the ear cartilage sheet pieces are allowed to enter the frame structure by repeated centrifugation.

14. Use of the ear cartilage tissue engineering complex of claim 1 for preparing a medical product for repairing joint defects.

15. A method for repairing joint defects, wherein the ear cartilage tissue engineering complex of claim 1 is used to be transplanted into the defect joint of a patient to be repaired.

16. The complex of claim 6, wherein the gelation culture is in vitro culture with a gelation medium containing the following components: high-glucose DMEM medium containing 4 to 5 wt % glucose, 10% FBS (v/v) and 100 U/ml penicillin-streptomycin.

17. The method of claim 11, wherein the ear cartilage gel comprises a cell population of chondrocytes and extracellular matrix secreted by chondrocytes, wherein the extracellular matrix encapsulates the cell population, and the ear cartilage gel is in a gel state, and the density of chondrocytes is at least 1.0×10⁸ cells/ml or 1.0×10⁸ cells/g.

18. The method of claim 11, wherein the ear cartilage sheet pieces comprise a cell population composed of chondrocytes and extracellular matrix secreted by chondrocytes, wherein the extracellular matrix encapsulates the cell population, and the ear cartilage pieces are prepared by mincing the ear cartilage sheet, wherein the density of ear chondrocytes is at least 1.0×10⁸ cells/ml or 1.0×10⁸ cells/g.
